(19) United States
(12) Patent Application Publication
(10) Pub. No.: US 2024/0260706 A1
(43) Pub. Date: Aug. 8, 2024

(54) Footwear Kit

(71) Applicant: Bared Trading Pty Ltd, Armadale (AU)

(72) Inventor: Anna Baird, Armadale (AU)

(21) Appl. No.: 18/395,806

(22) Filed: Dec. 26, 2023

Related U.S. Application Data
(63) Continuation of application No. 17/293,796, filed on May 13, 2021, now abandoned.

(30) Foreign Application Priority Data
Nov. 13, 2018 (AU) 2018101680

(51) Int. Cl. A43B 7/1464; A43B 7/1425; A43B 7/149; A43B 17/02

(52) U.S. Cl. CPC A43B 7/1464; A43B 7/1425; A43B 7/149; A43B 17/02

(57) ABSTRACT

A footwear kit, comprising: left and right items of footwear; left and right forefoot inserts receivable in the left and right footwear items, respectively, so as to tighten the fit thereof to a wearer; a fitting guide directing that, should the wearer have an orthotic for either or each foot, a said forefoot insert be arranged such that it underlies the orthotic in the footwear item for that foot if the fit of that footwear item with only the orthotic therein is loose.

The invention relates to footwear kits and associated methods of procuring or providing footwear and fitting it to a wearer.

Conventionally, insoles are used to increase support and cushioning and/or improve fit of footwear items. In general, insoles are purchased by the user separately from the footwear items, and as such, may not result in the most suitable fit for the particular user or the particular item of footwear. Users of orthotics especially may find it challenging to identify suitable insoles for those shoes in which they require an orthotic.

Alternatively, the user might visit a shoe store or consult a podiatrist to procure insoles for a specific pair of footwear items. It will be appreciated that this is a generally inconvenient, time-consuming and expensive process. Further, orders for footwear items are increasingly being placed remotely, eg via internet purchases. In this context, there is a need for kits and methods of procuring or providing footwear and fitting the footwear to a wearer that enable the fitting to be performed remotely.

According to an aspect of the present invention, there is provided a footwear kit, comprising:
left and right items of footwear:
left and right forefoot inserts receivable in the left and right footwear items, respectively, so as to tighten the fit thereof to a wearer:
a fitting guide directing that, should the wearer have an orthotic for either or each foot, a said forefoot insert be arranged such that it underlies the orthotic in the footwear item for that foot if the fit of that footwear item with only the orthotic therein is loose.

The kit may further comprise left and right footbeds for the left and right footwear items, respectively.

The left and right footbeds may be provided in the left and right footwear items, respectively, and the guide may direct that the footbed first be removed from the or each footwear item which is to receive a said orthotic.

The guide may additionally direct that, should the wearer have no orthotic for either or each foot, a respective said forefoot insert be arranged such that it underlies the footbed in the footwear item for that foot if the fit of that footwear item with only the footbed therein is loose.

The kit may comprise, in addition to said footbeds (“the first footbeds”), left and right footbeds (“further footbeds”) which are thinner than the first footbeds, wherein the guide additionally directs that, should the wearer have no orthotic for either or each foot, a said further footbed be inserted in the footwear item for that foot if the fit of that footwear item with a said first footbed therein is tight.

The left and right forefoot inserts may consist of a left forefoot insert and a right forefoot insert.

The left and right forefoot inserts may comprise left forefoot inserts of differing thickness and right forefoot inserts of differing thickness, whereby the fit of each footwear item to the wearer is variable, according to which of the respective inserts is inserted into that footwear item.

According to another aspect of the present invention, there is provided a method of procuring footwear and fitting it to a wearer having an orthotic for either or each foot, wherein:
procuring of the footwear comprises taking delivery of a kit which comprises:
   left and right items of said footwear; and
   left and right forefoot inserts receivable in the left and right footwear items, respectively, so as to tighten the fit thereof to a wearer; and
fitting of the footwear to the wearer comprises arranging, in the footwear item for the or each foot for which the wearer has a said orthotic, a said forefoot insert such that it underlies that orthotic, to determine whether the forefoot insert is required.

According to another aspect of the present invention, there is provided a method of providing and fitting footwear to a wearer having an orthotic for cither or each foot, the method comprising:
sending to the wearer a kit which comprises:
left and right items of said footwear; and
left and right forefoot inserts receivable in the left and right footwear items, respectively, so as to tighten the fit thereof to a wearer; and
directing the wearer to arrange, in the footwear item for the or each foot for which the wearer has a said orthotic, a said forefoot insert such that it underlies the orthotic in that footwear item if the fit of that footwear item with only the orthotic therein is loose.

The invention will now be described, by way of non-limiting example only, with reference to the accompanying drawings, in which:

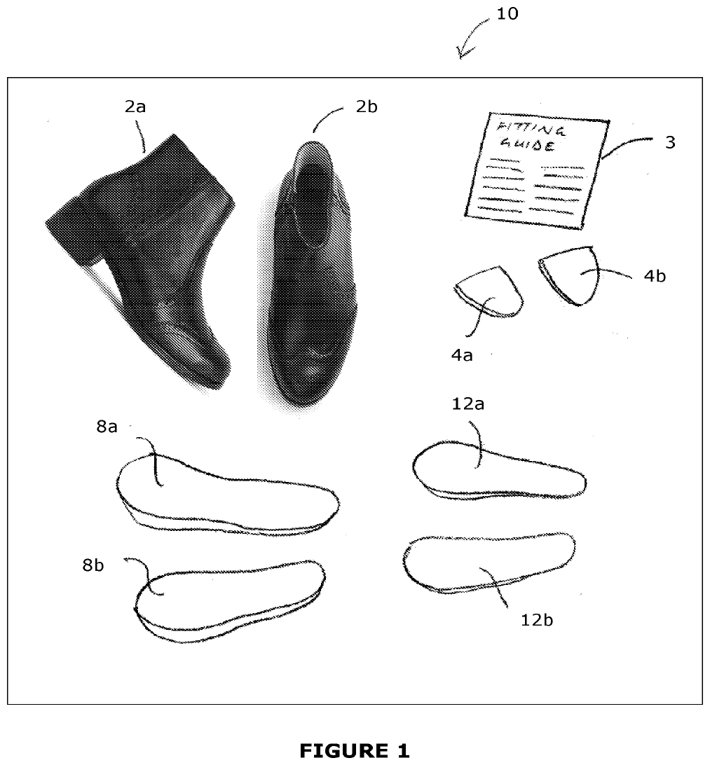

FIG. 1 is a schematic illustration of components of a footwear kit according to a preferred embodiment of the invention;

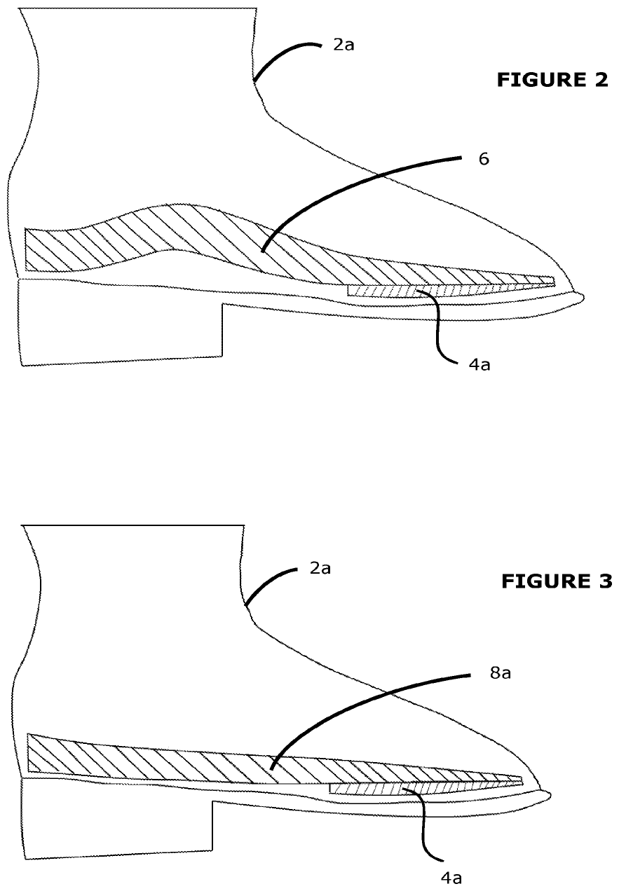

FIG. 2 is a schematic cross-sectional view of an item of footwear and a forefoot insert underlying an orthotic therein:

FIG. 3 is a schematic cross-sectional view of an item of footwear and a forefoot insert underlying a footbed therein; and

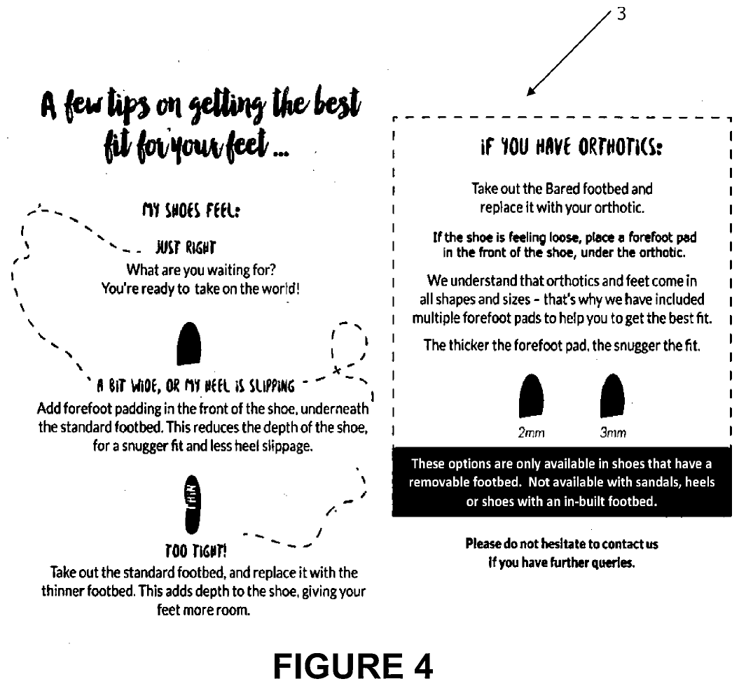

FIG. 4 illustrates a fitting guide.

A fitting kit 10 according to a preferred embodiment of the invention shown in FIG. 1, comprises left and right items of footwear 2a, 2b, left and right forefoot inserts 4a, 4b receivable in the left and right footwear items, respectively, so as to tighten the fit thereof to a wearer, and a fitting guide 3 directing that, should the wearer have an orthotic for either or each foot, a said forefoot insert 4a, 4b be arranged such that it underlies the orthotic in the footwear item for that foot if the fit of that footwear item with only the orthotic therein is loose. That is, for an orthotic user, the kit 10 enables the user to tighten the fit of the footwear item on the or each foot having an orthotic, by positioning the forefoot insert 4a, 4b under the orthotic 6, as illustrated in FIG. 2. The fitting guide 3 comprises written instructions, a preferred embodiment of which is shown in more detail in FIG. 4.

In a method of using the kit 10 in accordance with a preferred embodiment of the invention, the user firstly tries on the/each footwear item with only the orthotic therein. If the fit is loose, the user then, according to the fitting guide 3, inserts the forefoot insert under the orthotic in the footwear item in order to tighten the fit of the footwear item. If the fit of the footwear item with only the orthotic therein is comfortable, the user might be satisfied with the result, or might decide to try forefoot insert 4a, 4b under the orthotic and thereby compare the fit with and without the forefoot insert. In the case of the preferred embodiment, the orthotic 6 is the user's own orthotic. In another embodiment, the orthotic 6 is part of the kit 10.

The kit 10 further comprises left and right footbeds 8a, 8b for the left and right footwear items 2a, 2b respectively. The footbeds 8a, 8b are full-length footbeds, configured to be inserted into the footwear items to increase support and cushioning and/or improve fit. In some cases, the left and right footbeds 8a, 8b may be positioned/provided in the left and right footwear items 2a, 2b respectively when provided in the kit 10, and the guide 3 directs that the footbed 8a, 8b first be removed from the or each footwear item which is to receive the orthotic 6.

The guide 3 additionally directs that, should the wearer have no orthotic for either or each foot, a respective forefoot insert 4a, 4b be arranged such that it underlies the footbed 8a, 8b in the footwear item 2a, 2b for that foot if the fit of that footwear item with only the footbed 8a, 8b therein is loose. That is, where the user does not use orthotics (or if the user has an orthotic for only one foot, for the foot that does not have the orthotic), the kit 10 enables the user to tighten the fit of the footwear item(s) by positioning the forefoot insert 4a, 4b under the footbed 8a, 8b, as illustrated in FIG. 3.

The kit 10 comprises, in addition to the first footbeds 8a, 8b, further left and right footbeds 12a, 12b which are thinner than the first footbeds. Like the first footbeds 8a, 8b, the further footbeds 12a, 12b are full-length footbeds configured to be inserted into the footwear items. The guide additionally directs that, should the wearer have no orthotic for either or each foot, a further footbed 12a, 12b be inserted in the footwear item for that foot if the fit of that footwear item with the first footbed 8a, 8b therein is tight. That is, where the user does not use orthotics (or if the user has an orthotic for only one foot, for the foot that does not have the orthotic), the kit 10 enables the user to loosen the fit of the footwear item(s) by replacing the thicker footbed 8a, 8b with the thinner footbed 12a, 12b.

The kit 10 comprises a single left forefoot insert 4a and a single right forefoot insert 4b. The single forefoot inserts 4a, 4b may be identical or may differ for each foot. Alternatively, the kit may comprise a plurality of left forefoot inserts of differing thickness and a plurality of right forefoot inserts of differing thickness, whereby the fit of each footwear item to the wearer may be varied, according to which of the respective inserts is inserted into that footwear item. In this case, the guide may direct the user to try the different forefoot inserts under the orthotic 6 or under the footbed 8a, 8b in order to select the forefoot insert which provides the most satisfactory fit. In one example, two pairs of forefoot inserts may be provided, one set comprising left and right forefoot inserts of about 2 mm thick, and the other set comprising left and right forefoot inserts of about 3 mm thick.

The invention also relates to methods of procuring footwear and fitting it to a wearer having an orthotic for either or each foot. In one preferred embodiment of the invention, the method comprises procuring the footwear by taking delivery of a kit which comprises left and right items of the footwear 2a, 2b, and left and right forefoot inserts 4a, 4b receivable in the left and right footwear items, respectively, so as to tighten the fit thereof to the wearer, and fitting the footwear to the wearer by arranging, in the footwear item for the or each foot for which the wearer has the orthotic, a forefoot insert 4a, 4b such that it underlies that orthotic, to determine whether the forefoot insert is required. More specifically, during fitting, the wearer firstly tries on the footwear item with only the orthotic therein. If the fit is loose, the wearer inserts the forefoot insert under the orthotic in the footwear item in order to tighten the fit of the footwear item. If the fit (with only the orthotic therein) is comfortable, the wearer might be satisfied with the result, or might decide to try the footwear item with a forefoot insert 4a, 4b under the orthotic, and thereby compare fit with and without the forefoot insert.

In the preferred embodiment, procurement and fitting of the footwear is done remotely, ie away from a shop or foot clinic, and that fitting may be performed by the wearer without requiring a shop assistant or podiatrist. That is, the method involves the wearer ordering the items of footwear online, over the phone, by fax or by mail, receiving delivery of the items of footwear, trying on the footwear item on the or each foot for which the wearer has an orthotic, and adjusting the fit of that item of footwear if required, using the forefoot insert 4a, 4b provided in the kit.

The invention also relates to methods of providing and fitting footwear to a wearer having an orthotic for either or each foot. In one preferred embodiment of the invention, the method comprises sending to the wearer a kit which comprises left and right items of the footwear 2a, 2b, and left and right forefoot inserts 4a, 4b receivable in the left and right footwear items, respectively, so as to tighten the fit thereof to the wearer, and directing the wearer to arrange, in the footwear item for the or each foot for which the wearer has the orthotic, a forefoot insert 4a, 4b such that it underlies the orthotic in that footwear item, if the fit of that footwear item with only the orthotic therein is loose. In this example, the method comprises directing the wearer to firstly try on the footwear item 2a, 2b with only the orthotic therein. In the case that the fit is loose, the wearer is directed to insert the forefoot insert 4a, 4b under the orthotic in the footwear item in order to tighten the fit of the footwear item.

In the preferred embodiment, provision and fitting of the footwear to the wearer is performed remotely, ie away from a shop or foot clinic, and without requiring a shop assistant or podiatrist. That is, provision of the footwear involves sending the kit to the wearer in fulfilment of an order such as an online, phone, fax or mail order, and the directions for fitting the footwear to the wearer is by way of a written fitting guide provided as part of the kit.

In the foregoing methods, directions for fitting the footwear may be via written instructions in the form of an instruction card 3 or leaflet provided in the kit as described previously, or a written guide on a webpage. Alternatively, the directions may be via verbal instructions, or via pictorial, videographic or diagrammatic instructions.

The foregoing methods may additionally be applied for fitting footwear to a wearer having no orthotic for either or each foot, by directing the wearer to arrange a forefoot insert 4a, 4b such that it underlies the footbed 8a, 8b in the footwear item 2a, 2b for that foot if the fit of that footwear item with only the footbed 8a, 8b therein is loose. That is, where the user does not use orthotics (or if the user has an orthotic for only one foot, for the foot that does not have the orthotic), the method enables the user to tighten the fit of the footwear item(s) by positioning the forefoot insert 4a, 4b under the footbed 8a, 8b.

The method step of fitting footwear to a wearer having no orthotic for either or each foot may further comprise providing with the kit and in addition to the first footbeds 8a, 8b, further left and right footbeds 12a, 12b which are thinner than the first footbeds. Like the first footbeds 8a, 8b, the further footbeds 12a, 12b are full-length footbeds configured to be inserted into the footwear items. The method comprises directions that, should the wearer have no orthotic for either or each foot, a further footbed 12a, 12b be inserted in the footwear item for that foot if the fit of that footwear item with the first footbed 8a, 8b therein is tight. That is, where the user does not use orthotics (or if the user has an orthotic for only one foot, for the foot that does not have the orthotic), the method enables the user to loosen the fit of the footwear item(s) by replacing the thicker footbed 8a, 8b with the thinner footbed 12a, 12b.

The method step of fitting footwear to a wearer having no orthotic for either or each foot may further comprise, providing with the kit and in addition to the forefoot inserts 4a, 4b, a plurality of left forefoot inserts of differing thickness and a plurality of right forefoot inserts of differing thickness, whereby the fit of each footwear item to the wearer may be varied, according to which of the respective inserts is inserted into that footwear item. In this case, the fitting step may involve trying the different forefoot inserts under the orthotic 6 or under the footbed 8a, 8b in order to select the forefoot insert which provides the most satisfactory fit. In one example, two pairs of forefoot inserts may be provided, one set comprising left and right forefoot inserts of about 2 mm thick, and the other set comprising left and right forefoot inserts of about 3 mm thick.

While various embodiments of the present invention have been described above, it should be understood that they have been presented by way of example only, and not by way of limitation. It will be apparent to a person skilled in the relevant art that various changes in form and detail can be made therein without departing from the spirit and scope of the invention. Thus, the present invention should not be limited by any of the above described exemplary embodiments.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A footwear kit, comprising:
left and right items of footwear;
left and right forefoot inserts receivable in the left and right footwear items, respectively, so as to tighten the fit thereof to a wearer;
a fitting guide directing that, should the wearer have an orthotic for either or each foot, a said forefoot insert be arranged such that it underlies the orthotic in the footwear item for that foot if the fit of that footwear item with only the orthotic therein is loose.

2. A kit according to claim 1, further comprising left and right footbeds for the left and right footwear items, respectively.

3. A kit according to claim 2, wherein the left and right footbeds are provided in the left and right footwear items, respectively, and wherein the guide directs that the footbed first be removed from the or each footwear item which is to receive a said orthotic.

4. A kit according to claim 2, wherein the guide additionally directs that, should the wearer have no orthotic for either or each foot, a respective said forefoot insert be arranged such that it underlies the footbed in the footwear item for that foot if the fit of that footwear item with only the footbed therein is loose.

5. A kit according to claim 2, comprising, in addition to said footbeds (“the first footbeds”), left and right footbeds (“further footbeds”) which are thinner than the first footbeds, wherein the guide additionally directs that, should the wearer have no orthotic for either or each foot, a said further footbed be inserted in the footwear item for that foot if the fit of that footwear item with a said first footbed therein is tight.

6. A kit according to claim 1, wherein the left and right forefoot inserts consist of a left forefoot insert and a right forefoot insert.

7. A kit according to claim 1, wherein the left and right forefoot inserts comprise left forefoot inserts of differing thickness and right forefoot inserts of differing thickness, whereby the fit of each footwear item to the wearer is variable, according to which of the respective inserts is inserted into that footwear item.

8. A method of procuring footwear and fitting it to a wearer having an orthotic for either or each foot, wherein:
procuring of the footwear comprises taking delivery of a kit which comprises:
left and right items of said footwear; and
left and right forefoot inserts receivable in the left and right footwear items, respectively, so as to tighten the fit thereof to a wearer; and
fitting of the footwear to the wearer comprises arranging, in the footwear item for the or each foot for which the wearer has a said orthotic, a said forefoot insert such that it underlies that orthotic, to determine whether the forefoot insert is required.

9. A method of providing and fitting footwear to a wearer having an orthotic for either or each foot, the method comprising:
sending to the wearer a kit which comprises:
left and right items of said footwear; and
left and right forefoot inserts receivable in the left and right footwear items, respectively, so as to tighten the fit thereof to a wearer; and
directing the wearer to arrange, in the footwear item for the or each foot for which the wearer has a said orthotic, a said forefoot insert such that it underlies the orthotic in that footwear item if the fit of that footwear item with only the orthotic therein is loose.
